(19) United States
(12) Patent Application Publication
(10) Pub. No.: US 2008/0208306 A1
(43) Pub. Date: Aug. 28, 2008

(54) IMPLANTABLE MEDICAL LEAD WITH REVERSIBLE FIXATION MECHANISM

(76) Inventor: Jean J. G. Rutten, Bocholtz (NL)

Correspondence Address: MEDTRONIC, INC., 710 MEDTRONIC PARKWAY NE, MINNEAPOLIS, MN, 55432-9924, US

(21) Appl. No.: 11/679,262

(22) Filed: Feb. 27, 2007

(51) Int. Cl. A61N 1/00

(52) U.S. Cl. 607/127

(57) ABSTRACT

An implantable medical lead is provided, which includes an elongated lead body having a distal end portion and a pull cable lumen. A pull extends within the pull cable lumen and is fixedly coupled to the distal end portion. The pull cable is configured to be moved axially relative to the elongated lead body to form a fixation loop.

### TECHNICAL FIELD

This invention relates generally to an implantable medical device and, more particularly, to an implantable medical lead having a reversible fixation mechanism.

### BACKGROUND OF THE INVENTION

Pacemakers, implantable cardio defibrillators (ICDs), neurostimulators, and other implantable medical devices (IMDs) have been developed that may administer electrical therapy to an area in a patient's body. IMDs of this type typically include a pulse generator having a connector block to which one or more implantable medical leads are attached. During implantation, the pulse generator is disposed within a surgically-created pocket, and the distal end of the lead (or leads) is positioned adjacent the area to be treated. In the case of an ICD, for example, the distal end of the lead assembly may be positioned within one or more chambers of the heart (endocardial lead), on the surface of the heart (epicardial lead), or within the surrounding vasculature (transvenous lead). One or more fixation mechanisms anchor the distal end portion of the implantable medical lead in place. Exemplary distal fixation mechanisms include helical screws, tines, and various expandable structures (e.g., stent-like structures mounted around inflatable balloons). After the lead has been anchored at a desired location, site-specific electrical measurements are taken. The pulse generator is then programmed in accordance with the electrical measurements, and the IMD pocket is sutured closed to complete the operation.

Conventional fixation mechanisms of the type described above are limited in certain respects. For example, such fixation mechanisms may fail to retain the distal end portion of the implantable medical lead in the desired location for a prolonged period of time, especially if the lead is implanted in a turbulent area of the patient's body (e.g., the left ventricle). As a result, the lead may move after implantation, which may render inaccurate the electrical measurements utilized to program the pulse generator. As another limitation, many conventional fixation mechanisms are not readily reversible; that is, such fixation mechanisms may not be easily moved between anchored and unanchored states. Consequently, implantable leads employing such fixation mechanisms may be difficult to reposition after anchoring and to remove after implantation.

It should thus be appreciated that it would be desirable to provide an implantable medical lead having a reversible fixation mechanism. It would also be advantageous if such a fixation mechanism were capable of securely anchoring the distal end portion of the implantable medical lead at a desired location in a patient's vasculature. Other desirable features and characteristics of the present invention will become apparent from the subsequent detailed description of the invention and the appended claims, taken in conjunction with the accompanying drawings and this background of the invention.

### BRIEF DESCRIPTION OF THE DRAWINGS

The following drawings are illustrative of particular embodiments of the invention and therefore do not limit the scope of the invention, but are presented to assist in providing a proper understanding. The drawings are not to scale (unless so stated) and are intended for use in conjunction with the explanations in the following detailed descriptions. The present invention will hereinafter be described in conjunction with the appended drawings, wherein like reference numerals denote like elements, and:

FIGS. 1 and 2 are side cross-sectional views of an implantable medical lead in an unanchored and anchored state, respectively, in accordance with a first exemplary embodiment of the present invention.

FIG. 3 is an isometric cross-sectional view of the implantable medical lead shown in FIGS. 1 and 2 taken along line 3-3 (FIG. 2).

FIGS. 4 and 5 are side cross-sectional views of an implantable medical lead in an unanchored and anchored state, respectively, in accordance with a second exemplary embodiment of the present invention.

FIG. 6 is a side cross-sectional view of an exemplary proximal connector including a plurality of flexible bellows.

FIGS. 7 and 8 are side cross-sectional views of the distal end portion of an implantable medical lead in an unanchored and anchored state, respectively, in accordance with a third exemplary embodiment of the present invention.

FIG. 9 is a top plan view of the distal end portion of the implantable medical lead shown in FIGS. 7 and 8.

FIGS. 10 and 11 are side cross-sectional views of the distal end portion of an implantable medical lead in an unanchored and anchored state, respectively, in accordance with a fourth exemplary embodiment of the present invention.

FIG. 12 is an isometric cross-sectional view of the distal end portion of the implantable medical lead shown in FIGS. 10 and 11 wherein the lead body includes two integrally-formed tubes.

FIG. 13 is an isometric cross-sectional view of the distal end portion of the implantable medical lead shown in FIGS. 10 and 11 wherein the lead body includes two tubes joined together by an adhesive.

### DETAILED DESCRIPTION OF AT LEAST ONE EXEMPLARY EMBODIMENT

The following description is exemplary in nature and is not intended to limit the scope, applicability, or configuration of the invention in any way. Rather, the following description provides a convenient illustration for implementing various exemplary embodiments of the present invention. Various changes to the described embodiments may be made in either the function or the arrangement of the elements described herein without departing from the scope of the invention.

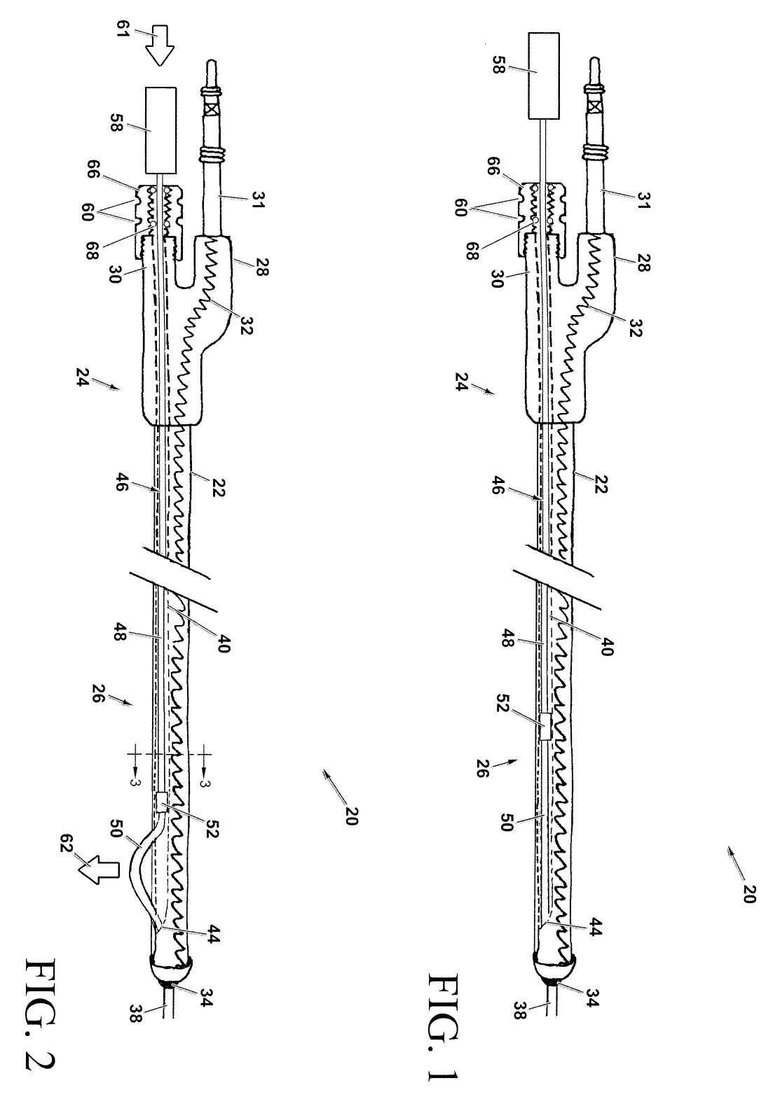

FIGS. 1 and 2 are side cross-sectional views of an implantable medical lead 20 in an unanchored and anchored state, respectively, in accordance with a first exemplary embodiment of the present invention. Lead 20 includes an elongated lead body 22 having a proximal end portion 24 and a distal end portion 26. Lead body 22 may be made of a flexible, biocompatible material, such as silicon rubber, polyurethane, or the like. In the illustrated exemplary embodiment, the proximal end of proximal end portion 24 is bifurcated into a first leg 28 and a second leg 30. First leg 28 may include, for example, a connector 31 (e.g., a standardized IS1 connector), which may be plugged into the connector block of a non-illustrated pulse generator. At least one electrode 34 (e.g., a distal tip electrode) is disposed on distal end portion 26. Electrode 34 is electrically coupled to connector 31 by way of at least one filar 32, which runs axially within lead body 22.

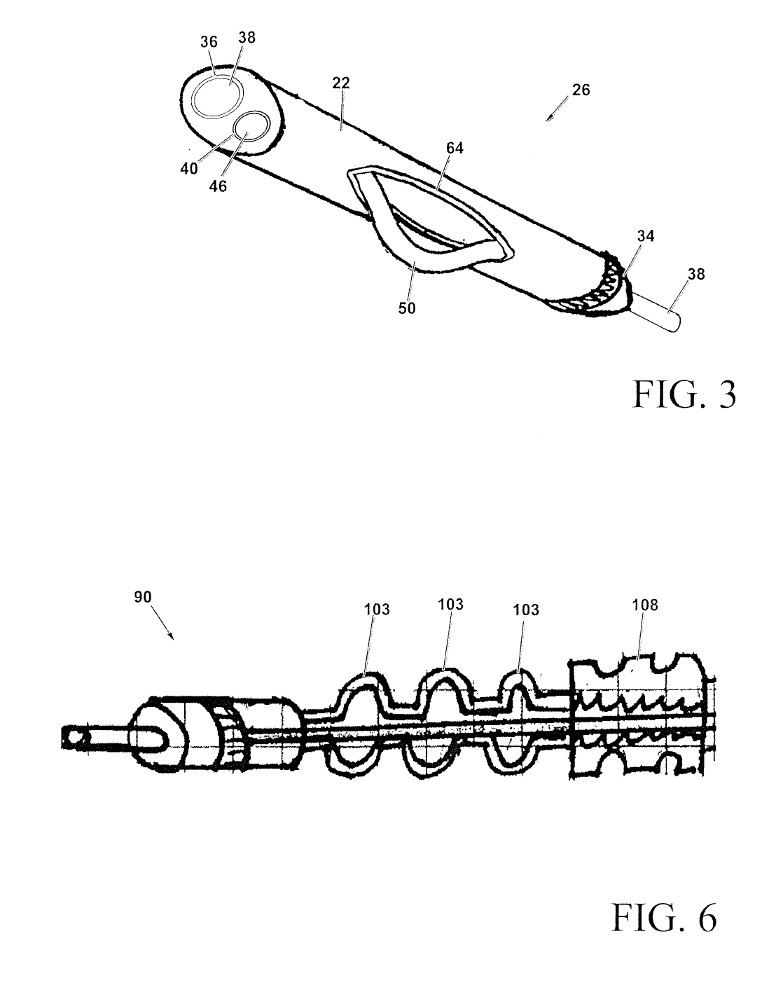

FIG. 3 is an isometric cross-sectional view of distal end portion 26 of lead body 22 taken along line 3-3 (FIG. 2). As can be seen in FIG. 3, a guide wire lumen 36 extends axially within lead body 22. Filar 32 may be coiled around guide wire lumen 36 in the well-known manner. Guide wire lumen 36 is sized to receive a guide wire 38 therethrough. In the illustrated exemplary embodiment, implantable medical lead 20 is depicted as an over-the-wire lead; that is, implantable medical lead 20 is configured to be advanced over guide wire 38 after guide wire 38 has been maneuvered to the implantation site. In other embodiments, however, it should be appreciated that implantable medical lead 20 may be another type of lead, such as a rapid exchange lead. In addition, it should be understood that implantable medical lead 20 may be configured to be implanted at any suitable location in a patient's body. For example, implantable medical lead 20 may be a left ventricle lead, a coronary sinus lead, a cardiac vein lead, a trans-esophageal pacing lead, a coronary sinus/cardiac vein ablation catheter, or the like.

In addition to guide wire lumen 36, lead body 22 includes a pull cable lumen 40, which runs axially within lead body 22. Pull cable lumen 40 extends through proximal end portion 24 and to an inner wall 44 provided within distal end portion 26 of lead body 22. A pull cable 46 resides within pull cable lumen 40 and may slide therein. Pull cable 46 includes two segments: an elongated pull cable body 48, and a flexible distal segment 50. Flexible distal segment 50 is preferably formed from a flexible material (e.g., silicon rubber), and pull cable body 48 is preferably formed from a biocompatible metal or alloy, such as that utilized to form filar 32. In certain embodiments, pull cable 48 may comprise a metal core clad in an insulative sheathing (e.g., polyetheretherketone). The distal end of pull cable body 48 is joined to the proximal end of flexible distal segment 50. For example, a crimping sleeve 52 may be welded or crimped to the proximal end of pull cable body 48 and adhesively bonded to the distal end of flexible distal segment 50. At its distal end, flexible distal segment 50 (and thus pull cable 46) is fixedly coupled to lead body 22; e.g., the distal tip of flexible distal segment 50 may be embedded within an inner wall 44 of lead body 22 as indicated in FIGS. 1-3.

The proximal end of pull cable 46 extends through an opening provided in leg 30. A handle 58 may be fixedly coupled to the proximal end of pull cable 46. A user (e.g., a surgeon) may push or pull handle 58 to move pull cable 46 axially relative to elongated lead body 22. When handle 58 is in the position shown in FIG. 1, flexible distal segment 50 remains within pull cable lumen 40. When pull cable 46 remains in this unanchored state, elongated lead body 22 may be easily maneuvered through tortuous vascular pathways. In contrast, when handle 58 is pushed toward proximal end portion 24 (indicated by arrow 61 in FIG. 2), pull cable 46 slides distally, which causes flexible distal segment 50 to bulge radially outward (indicated by arrow 62 in FIG. 2) and thereby form a fixation loop proximate distal end portion 26 of implantable medical lead 20. As shown in FIG. 3, an opening 64 (e.g., a longitudinal slit) in lead body 22 permits flexible distal segment 50 to protrude through the outer wall of lead body 22. When implantable lead body 20 is advanced to a treatment site within a patient's vasculature, the distal fixation loop abuttingly contacts an anatomical wall or other such feature to anchor distal end portion 26 in place. This arched position of pull cable 46 (i.e., the position in which the fixation loop is formed) is thus generally referred to as the anchored state. The surgeon may adjust the size of the fixation loop, and thus the intra-vascular clamping force of implantable medical lead 20, by adjusting the position of handle 58 relative to distal end portion 26.

To secure pull cable 46 in the anchored state, implantable medical lead 20 may be provided with a locking mechanism. In the illustrated embodiment, implantable medical lead 20 is provided with a clamping body 66, which is threadably coupled to the proximal end of leg 30. Rotation of clamping body 66 in a first direction compresses leg 30 around a proximal segment of pull cable 46 to retain pull cable 46 in a desired position. Clamping body 66 may thus be utilized to lock pull cable 46 in the anchored state and thereby maintain the distal fixation loop formed by flexible distal segment 50. Furthermore, suture features 60 permit clamping body 66 to be sutured to a patient's body after pull cable 46 has been locked in a desired position. By permitting pull cable 46 to be locked in the anchored state and clamping body 66 to be sutured, implantable medical lead 20 ensures that distal end portion 26 remains securely anchored at desired location.

To prevent the ingress of bodily fluids into pull cable lumen 40, clamping body 66 may include one or more sealing features 68 (e.g., polymeric o-rings), which sealingly engage the inner surface of pull cable 46. In addition, a sealing body may be disposed proximate opening 64 and coupled to pull cable 46. For example, as shown in FIGS. 1-3, crimp sleeve 52 may be disposed proximate opening 64 and sealingly engage an inner surface of pull cable lumen 40. In other embodiments, the sealing body may take the form of an annular collar, an electrode, or simply a portion of pull cable 46 having an outer diameter substantially equivalent to the inner diameter of pull cable lumen 40.

Unlike many of the conventional fixation mechanisms described above, the distal fixation loop formed by pull cable 46 securely anchors implantable medical lead 20 at a desired location in a patient's vasculature, consequently decreasing the likelihood that implantable medical lead 20 will move after implantation. In addition, the distal fixation mechanism of implantable medical lead 20 is reversible; i.e., pull cable 46 may be readily moved between the anchored and unanchored positions by simply pushing or pulling handle 58, respectively. In this manner, implantable medical lead 20 permits a surgeon to anchor, unanchor, reposition, and re-anchor lead 20 as often as desired during the implantation process. Furthermore, if it later becomes desirable to remove lead 20 (e.g., due to infection), a surgeon may simply open the IMD pocket, pull on handle 58 to move pull cable 46 to its unanchored state, and withdraw lead 20.

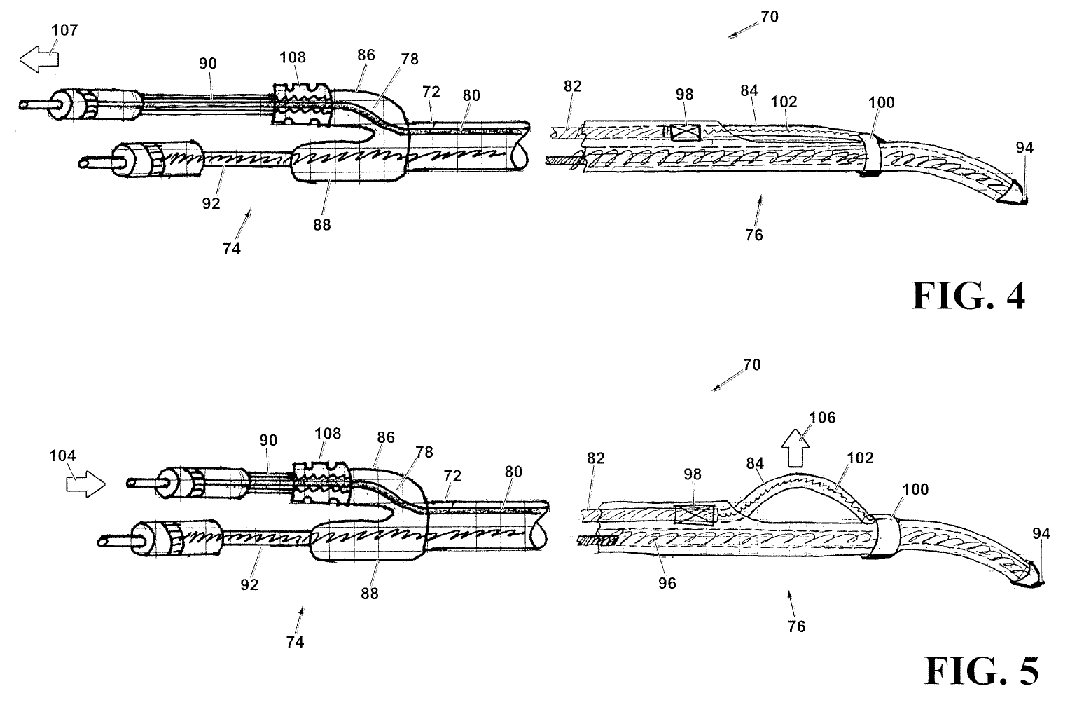

The foregoing has thus described the manner in which pull cable 46 permits a user (e.g., a surgeon) to anchor and unanchor distal end portion 26 from the proximal end of implantable medical lead 20. In certain embodiments of the present invention, pull cable 46 may also serve a second function; i.e., pull cable may be utilized to electrically couple one or more distal electrodes to a proximal electrical source (e.g., a pulse generator). To further illustrate this point, FIGS. 4 and 5 are side cross-sectional views of an implantable medical lead 70 in unanchored and anchored states, respectively, in accordance with a second exemplary embodiment of the present invention. As may be appreciated by comparing FIGS. 4 and 5 to FIGS. 1-3, implantable medical lead 70 is similar to lead 20 in several respects. For example, implantable medical lead 70 includes an elongated lead body 72 having a proximal end portion 74 and a distal end portion 76. A pull cable lumen 78 and a guide wire lumen (not shown) extend through lead body 72 from proximal end portion 74 to distal end portion 76. A pull cable 80 resides within pull cable lumen 78 and may slide axially therein. Pull cable 80 includes an elongated pull cable body 82 (e.g., a biocompatible metal or alloy) and a flexible distal segment 84 (e.g., silicon rubber). The proximal end of flexible distal segment 84 may be joined to the distal end of pull cable body 82 via a crimp sleeve (not shown), such as crimp sleeve 52 described above in conjunction with FIGS. 1-3. However, unlike the pull cable lumen of implantable medical lead 20, pull cable lumen 78 is truncated such that flexible distal segment 84 resides outside of pull cable lumen 78 when in the unanchored position (FIG. 4).

As was the case previously, proximal end portion 74 is bifurcated into a first leg 86 and a second leg 88. However, in this particular case, legs 86 and 88 both include a connector (i.e., connector 90 and connector 92, respectively), such as a standardized IS1 connector. Connector 92 is electrically coupled to a tip electrode 94 mounted on the distal tip of lead body 72 by way of a filar 96. Connector 90 is electrically coupled to pull cable 80, which is, in turn, electrically coupled to at least one electrode disposed on cable 80 or lead body 72. For example, as shown in FIGS. 4 and 5, pull cable 80 may be electrically coupled to a first ring electrode 98 mounted on cable 80 and a second ring electrode 100 mounted around distal end portion 76. If flexible distal segment 84 is made of a non-conductive material (e.g., silicon rubber), a filar 102 may extend through segment 84 to electrically couple pull cable body 82 to ring electrode 100.

The body of connector 90 is preferably formed from an elastic material (e.g., silicon rubber, polyurethane, etc.) that may withstand the strain of being stretched and compressed. Also, in certain embodiments, the body of connector 90 may such movement by forming a series of flexible bellows 103 when axially compressed as shown in FIG. 6. In this manner, connector 90 is adapted to be pulled or pushed by a surgeon to move pull cable 80 axially relative to lead body 72 so as to deploy or retract a distal fixation loop. More specifically, when connector 90 is pushed in the direction of arrow 104 (FIG. 5), pull cable 80 slides distally thus causing flexible distal segment 84 to bulge radially outward and form a distal fixation loop (indicated by arrow 106 FIG. 5). Conversely, when connector 90 is pulled in the direction of arrow 107 (FIG. 4), pull cable 80 slides proximally thus returning flexible distal segment 84 to its non-protruding state and retracting the distal fixation loop. After pull cable 80 has been moved to a desired position, a clamping body 108 may be utilized to secure pull cable 80 in the manner described above. Connectors 90 and 92 may then be plugged into the connector block of a non-illustrated pulse generator, and operation may be completed.

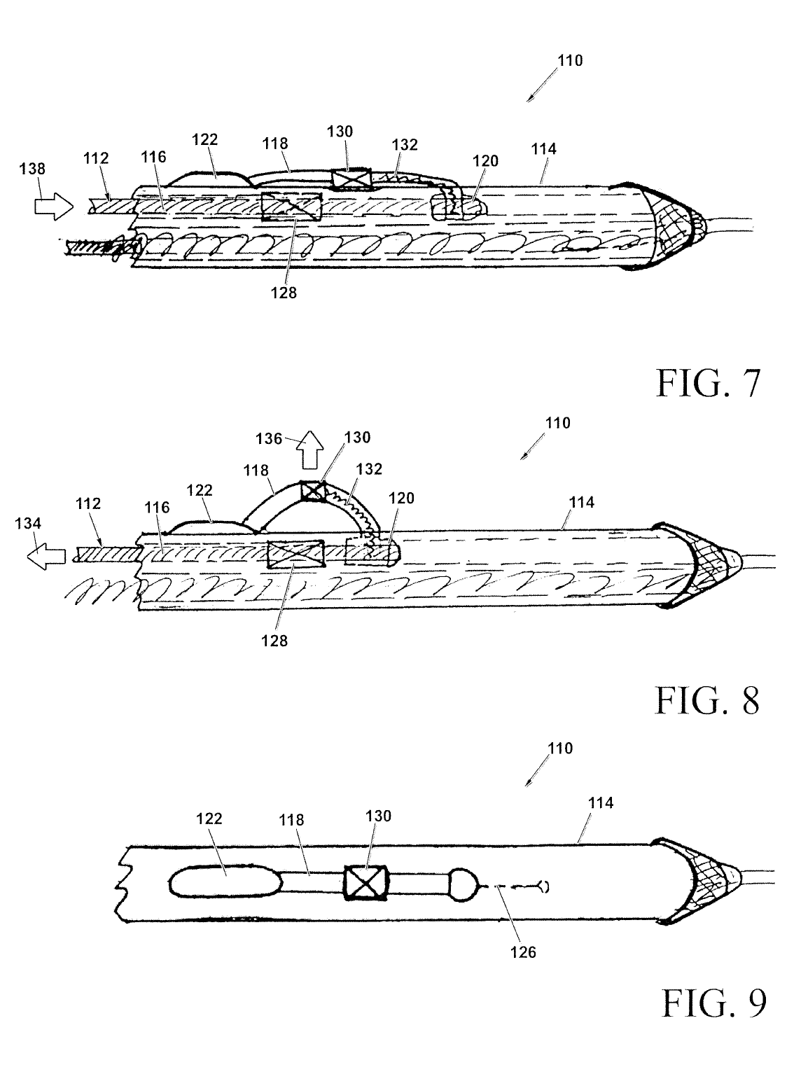

The foregoing has described two exemplary implantable medical leads wherein the distal fixation mechanism is moved to between its anchored and unanchored states by pushing and pulling, respectively, the proximal end of a pull cable, whether the proximal end is attached to a handle (e.g., handle 58 shown in FIGS. 1 and 2) or a connector (e.g., connector 90 shown in FIGS. 4 and 5). By comparison, FIGS. 7 and 8 are side cross-sectional views of the distal end portion of an implantable medical lead 110 that may be moved between its anchored state (FIG. 8) and its unanchored state (FIG. 7) by pulling and pushing, respectively, the proximal end of a pull cable 112. Implantable medical lead 110 is further illustrated in FIG. 9, which is a top plan view of the distal end portion of lead 110. Although not shown in FIGS. 7-9, it should be appreciated that the proximal portion end of implantable medical lead 110 may resemble that of implantable medical lead 70 (FIGS. 4 and 5).

Referring now to FIGS. 7-9, implantable medical lead 110 includes an elongated lead body 114, and pull cable 112 includes an elongated pull cable body 116 and a flexible distal segment 118. The distal end of flexible distal segment 118 is coupled the distal end of pull cable body 116 by way of, for example, a crimp sleeve 120. The proximal end of flexible distal segment 118 is coupled to an outer surface of elongated lead body 114; e.g., the proximal end of flexible distal segment 118 may be attached (e.g., adhered) to a bulge or protrusion 122 provided on elongated lead body 114. As shown in FIG. 9, flexible distal segment 118 passes through an opening 126 (e.g., a longitudinal slit) provided in elongated lead body 114. In the illustrated exemplary embodiment, pull cable 112 is electrically coupled to first and second ring electrodes 128 and 130. Ring electrode 128 is mounted around elongated pull cable body 116 proximate opening 126, and ring electrode 130 is mounted around flexible distal segment 118. If flexible distal segment 118 is made of a non-conductive material, ring electrode 130 may be electrically coupled to pull cable body 116 by way of a filar 132 disposed within flexible distal segment 118.

When pull cable 112 is pulled (indicated by arrow 134 in FIG. 8), flexible distal segment 118 forms a distal fixation loop proximate the distal end portion of implantable medical lead 110 (indicated by arrow 136 in FIG. 8) to anchor lead 110 in a desired location. Note that ring electrode 130 is positioned on flexible distal segment 118 such that electrode 130 is located at the apex of the distal fixation loop. Positioning ring electrode 130 in this manner permits electrode 130 to contact an inner wall of the patient's vasculature, which may ultimately improve the effectiveness of the electrical therapy delivered by electrode 130 and, more generally, implantable medical lead 110. To retract the distal fixation loop and unanchor lead 110, pull cable 112 is simply pushed toward the distal end of implantable medical lead 110 in the manner indicated by arrow 138 (FIG. 7).

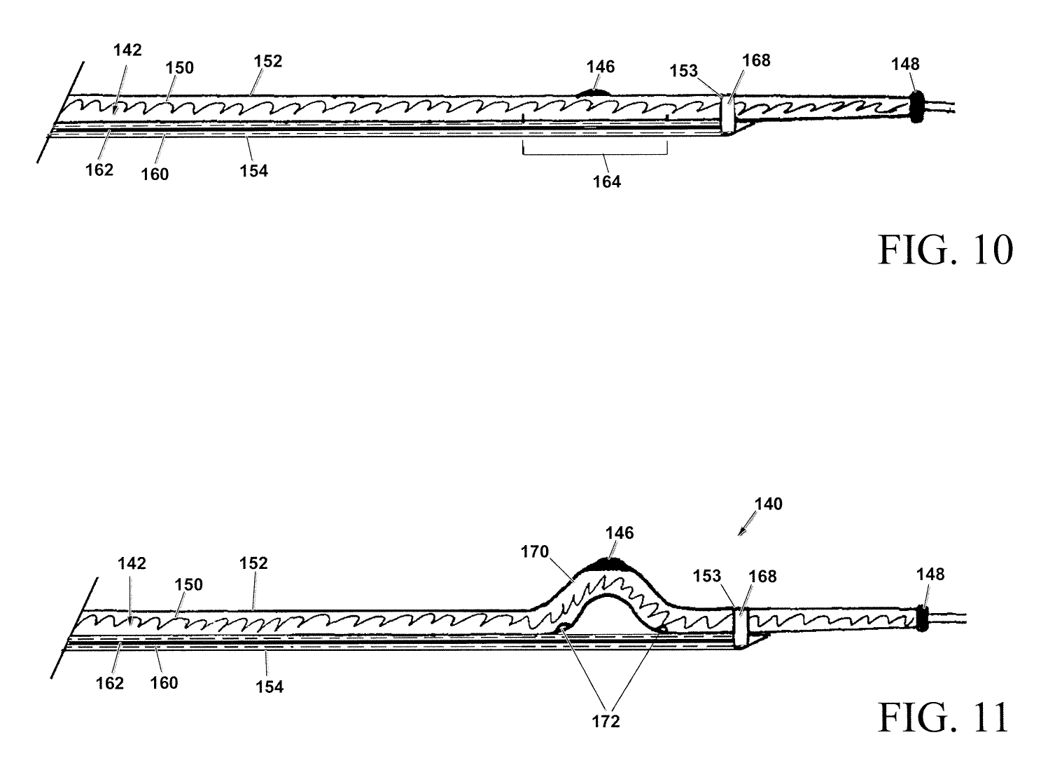

Considering the foregoing, it should be appreciated that three exemplary implantable medical leads have thus been described wherein the pull cable is configured to slide axially within an elongated lead body to form a distal fixation loop proximate the lead body's distal end portion. These examples notwithstanding, it should further be understood that the implantable medical lead may also be configured such that the elongated lead body bulges radially outward to form one or more distal fixation loops. FIGS. 10 and 11 are cross-sectional views of the distal end portion of such an implantable medical lead 140 in its unanchored (FIG. 10) and anchored (FIG. 11) states. Lead 140 includes an elongated lead body 142 (only the distal end portion of which is shown in FIGS. 10 and 11), which carries a first electrode 146 and a second electrode 148 (e.g., a distal tip electrode). Electrodes 146 and 148 are coupled to a proximal connection (not shown) by way of a plurality of filars 150, which extend axially within elongated lead body 142. In the illustrated exemplary embodiment, elongated lead body 142 is formed from first and second tubular bodies. As described more fully below in conjunction with FIG. 12, the second tubular body is longitudinally coupled to the first tubular body with the exception of one or more splits area.

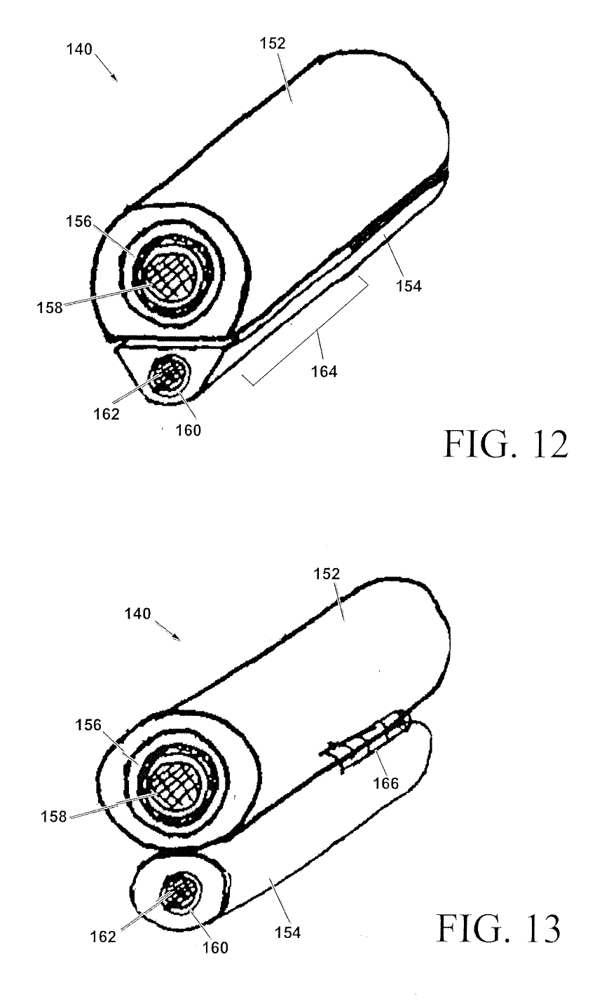

FIG. 12 is a cross-sectional isometric view of implantable medical lead 140. In this view, it can be seen that implantable medical lead 140 comprises an upper tube 152 and a lower tube 154. Upper tube 152 includes a guide wire lumen 156 through which a guide wire 158 may extend. Similarly, lower tube 154 includes a pull cable lumen 160 through which a pull cable 162 may extend. As indicated above, lower tube 154 may be longitudinally attached to upper tube 152 with the exception of split area 164 (FIGS. 10 and 12). Upper tube 152 and lower tube 154 may be formed as single, integral body, which is subsequently cut along split area 164. Alternatively, tubes 152 and 154 may be formed as separate bodies, which are subsequently joined together by an adhesive 166 as shown in FIG. 13. In the exemplary embodiment illustrated in FIGS. 10-12, implantable medical lead 140 includes one such split area 164. As described below, split area 164 permits upper tube 152 to arch away from lower tube 154 and form a distal fixation loop when pull cable 162 is pulled in the proximal direction. This example not withstanding, it should be appreciated that other embodiments may include two or more split areas, which permit the formation of two or more fixation loops proximate the distal end portion of implantable medical lead 140.

As was the case previously, pull cable 162 is fixedly coupled to elongated lead body 142 at the distal end portion thereof. This may be accomplished by way of, for example, a crimp sleeve or an annular bus 168, which may be made of a biocompatible metal or alloy. Pull cable 162 may be bonded (e.g., welded or soldered) to bus 168, which may be crimped around upper tube 152 at an attachment point 153 distal of split area 164. When pulled in the proximal direction, pull cable 162 slides axially within lower tube 154 and pulls bus 168 proximally. Bus 168, in turn, pulls upper tube 152 proximally at attachment point 153. As a result, the portion of upper tube 152 that is not longitudinally coupled to lower tube 154 (i.e., the portion of upper tube 152 corresponding to split area 164) bulges away from lower tube 154 to form a distal fixation loop 170 (FIG. 11). Again, to promote electrode-wall contact, electrode 146 may be disposed on the apex of fixation loop 170. Furthermore, as shown in FIG. 11 at 172, upper tube 152 may include one or more “hinged” areas (i.e., areas of reduced thickness) proximate the ends of split area 164 to facilitate the bending of upper tube 152 and, therefore, the formation of fixation loop 170.

There has thus been provided an implantable medical lead having a reversible fixation mechanism capable of securely anchoring the distal end portion of the lead in a desired location. Although the invention has been described with reference to a specific embodiment in the foregoing specification, it should be appreciated that various modifications and changes can be made without departing from the scope of the invention as set forth in the appended claims. Accordingly, the specification and figures should be regarded as illustrative rather than restrictive, and all such modifications are intended to be included within the scope of the present invention.

1. An implantable medical lead, comprising:
an elongated lead body having a distal end portion and a pull cable lumen; and
a pull cable extending within the pull cable lumen and fixedly coupled to said distal end portion, said pull cable configured to be moved axially relative to said elongated lead body to form a fixation loop.

2. An implantable medical lead according to claim 1, wherein said fixation loop is formed proximate said distal end portion.

3. An implantable medical lead according to claim 1, wherein said fixation loop is formed by said elongated lead body.

4. An implantable medical lead according to claim 3, further comprising an electrode disposed on said elongated lead body proximate said fixation loop.

5. An implantable medical lead according to claim 4, wherein said electrode is disposed on the apex of said fixation loop.

6. An implantable medical lead according to claim 3, wherein said elongated lead body comprises:
a first tube having a guide wire lumen therein;
a second tube having said pull cable lumen therein and longitudinally coupled to said first tube; and
a split area between said first tube and said second tube proximate said fixation loop.

7. An implantable medical lead according to claim 6, wherein said pull cable is fixedly coupled to said first tube proximate said distal end portion.

8. An implantable medical lead according to claim 1, wherein said pull cable comprises:
an elongated cable body; and
a flexible distal segment coupled to said elongated cable body, said flexible distal segment configured to form said fixation loop.

9. An implantable medical lead according to claim 8, wherein the proximal end of said flexible distal segment is fixedly coupled to the distal end of said elongated cable body, and wherein the distal end of said flexible distal segment is fixedly coupled to said elongated lead body.

10. An implantable medical lead according to claim 8, wherein the distal end of said flexible distal segment is fixedly coupled to the distal end of said elongated cable body, and wherein the proximal end of said flexible distal segment is fixedly coupled to an outer surface of said elongated lead body.

11. An implantable medical lead according to claim 8, wherein said elongated lead body comprises a proximal end portion including a connector, and wherein the implantable medical lead further comprises an electrode disposed on said pull cable, said pull cable electrically coupling said connector to said electrode.

12. An implantable medical lead according to claim 11, wherein said electrode is disposed on said flexible distal segment.

13. An implantable medical lead according to claim 1, wherein said elongated lead body comprises a proximal end portion through which said pull cable extends, and wherein the implantable medical lead further comprises a locking mechanism coupled to said proximal end portion and configured to secure said pull cable in a desired position.

14. An implantable medical lead according to claim 14, wherein said locking mechanism comprises a clamping body threadably coupled to said proximal end portion.

15. An implantable medical lead, comprising:
an elongated lead body having a proximal end portion, a distal end portion, and a pull cable lumen;
a pull cable disposed within said pull cable lumen and fixedly coupled to said distal end portion, said pull cable configured to be moved axially relative to said elongated lead body between (i) an unanchored position and (ii) an anchored position wherein a fixation loop is formed proximate said distal end portion; and
a locking mechanism coupled to said proximal end portion and configured to secure said pull cable in the anchored position.

16. An implantable medical lead according to claim 15, wherein said pull cable includes a flexible distal segment adapted to form said fixation loop.

17. An implantable medical lead according to claim 15, wherein said proximal end portion is bifurcated into a first leg and a second leg, said first leg including a connector.

18. An implantable medical lead according to claim 17, wherein said pull cable is electrically coupled to said connector, and wherein said pull cable is configured to be moved axially by adjusting the position of said connector relative to said elongated lead body.

19. An implantable medical lead, comprising:
an elongated lead body having a pull cable lumen and a distal end portion; and
a pull cable extending within the pull cable lumen, said pull cable comprising:
an elongated cable body configured to slide axially relative to said elongated lead body; and
a flexible distal segment coupled between said elongated cable body and said distal end portion, said flexible distal segment configured to be moved between an anchored position and an unanchored position by the axial sliding of said elongated cable body.

20. An implantable medical lead according to claim 19, wherein said elongated lead body further comprising a proximal connector, and wherein the implantable medical lead further comprises an electrode mounted on said flexible distal segment and electrically coupled to said proximal connector.
